(19) United States
(12) Patent Application Publication
(10) Pub. No.: US 2026/0284040 A1
(43) Pub. Date: Sep. 24, 2026

(54) COMBINATION OF A PARP1 INHIBITOR AND A SELECTIVE ESTROGEN DEGRADER FOR TREATING CANCER

(71) Applicant: ASTRAZENECA AB, Södertälje (SE)

(72) Inventors: Mark Robert ALBERTELLA, Cambridge (GB); Sabina Chiara COSULICH, Cambridge (GB); Jessica S. BROWN

(21) Appl. No.: 19/474,229

(22) PCT Filed: May 10, 2024
(86) PCT No.: PCT/EP2024/062952

§ 371 Date: Oct. 10, 2025

Related U.S. Application Data
(60) Provisional application No. 63/501,425, filed on May 11, 2023.

(51) Int. Cl. A61K 31/496; A61K 31/444; A61P 35/00

(52) U.S. Cl. CPC A61K 31/496; A61K 31/444; A61P 35/00

(57) ABSTRACT

A method of treating cancer in a subject in need thereof, comprising administering to the subject a first amount of a selective PARP1 inhibitor or a pharmaceutically acceptable salt thereof, and a second amount of a selective estrogen degrader or a pharmaceutically acceptable salt thereof, wherein the first amount and the second amount together comprise a therapeutically effective amount.

The present disclosure relates to methods of treating cancer, and in particular breast cancer such as ER positive breast cancer, in a patient in need thereof.

### BACKGROUND

Breast cancer remains one of the most significant public health problems, with an increasing global incidence (Sharma 2019, Chen 2022). It is the most common cancer among women, representing 24.5% of all newly diagnosed cases, and is currently the leading cause of cancer death among women worldwide, accounting for 15.5% of all cancer deaths (Sung 2021). Breast cancer is a highly heterogeneous disease, distinguished by different molecular subtypes, risk factors, clinical behaviours, and responses to treatment (Lüönd 2021). Of the clinically annotated subtypes, hormone receptor (HR)-positive breast cancers are the most common (Stravodimou 2020), characterized by positive immunohistochemical staining for the estrogen receptor (ER) and/or the progesterone receptor (PR) (Alisson 2020). Approximately 70-80% of all diagnosed breast cancers are ER-positive (ER+) (Stravodimou 2020, Harvey 1999, Lumachi 2015), of which 65% are also PR-positive (Lumachi 2015). Since 2007, there has been a steady increase in incidence rates of ER-positive breast cancer in Western countries, possibly linked to the obesity epidemic, and more widespread mammographic screening which preferentially detects slow-growing ER-positive cancers (Sung 2021).

Estrogen Receptor alpha (ERα, also known as NR3A1) is a member of the steroid hormone receptor super family that is encoded by the gene ESR1. Upon binding by estrogens, such as estradiol, ERα binds to estrogen responsive elements present in the promoter and/or enhancer regions of target genes and recruits specific co-factors which initiate or inhibit transcription. The ERα is a well-established drug target in breast cancer due to its involvement in the pathogenesis of breast tumours, with anti-endocrine therapies being the mainstay of treatment (Cardoso 2017). Currently, the selective estrogen receptor degrader (SERD) fulvestrant is used as a standard of care treatment for hormone receptor positive, HER2-negative metastatic breast cancer. Fulvestrant induces the degradation of ERα as well as antagonising ERα and ERB. Although fulvestrant has demonstrated superior clinical efficacy to other endocrine therapies in this setting, it is not orally bioavailable and the monthly intra-muscular route of administration may limit its efficacy (Robertson 2007).

Camizestrant, also known as AZD9833, is an orally bioavailable, potent, next generation selective ER antagonist and degrader (SERD) being developed for the treatment of ER-positive breast cancer. Its synthesis is described in Scott 2020. In addition to the degradation of Estrogen Receptor (ERα), camizestrant also acts as a pure ER antagonist. Camizestrant has the potential to further inhibit ERα activity and deliver greater OS in advanced breast cancer patients than currently available therapies targeting this pathway. Camizestrant has demonstrated antitumour activities in a wide range of ER+ cell lines and PDX models, which include models with mutations in the ESR1 gene (ESR1m) that encodes mutant ERα.

Tumors exhibiting homologous recombination deficiency (HRD) have traditionally been treated with targeted therapies against BRCA1/2. HRD is defined as the inability of a cell to effectively repair DNA double-strand breaks using the homologous recombination repair (HRR) pathway. Deficiencies in the HRR pathway have been identified in multiple tumor types, including breast, ovarian, and prostate cancers. Affected HRR genes associated with the HRD phenotype include BRCA1/2, ATM, PALB2, and RAD51 (Stewart 2022).

In breast cancer, mutations in BRCA1/2 have been strongly linked with HRD, and the development of HRD can contribute to the growth of breast cancer. However, there are patients with BRCA1/2 wild-type breast cancer that also displays HRD. Chemotherapy-based therapeutic strategies and PARP inhibitors have shown some efficacy in targeting the HRD, with the latter gaining significant popularity in the space because of the lack of toxicity that typically accompanies treatment with chemotherapy (denBrok 2017).

Two PARP inhibitors are approved by the FDA for patients with breast cancer: talazoparib (Talzenna) and olaparib (Lynparza).

While much progress has been made in the treatment of ER-positive breast cancer, many patients who have such cancers live with an incurable disease. Accordingly, it is important to continue to find new treatments for patients with incurable cancer.

### SUMMARY

In some embodiments, disclosed is a method of treating cancer in a subject in need thereof, comprising administering to the subject a first amount of a selective PARP1 inhibitor or a pharmaceutically acceptable salt thereof, and a second amount of a selective estrogen degrader or a pharmaceutically acceptable salt thereof. In the method, the first amount and the second amount together comprise a therapeutically effective amount.

In some embodiments, disclosed is a selective PARP1 inhibitor, or a pharmaceutically acceptable salt thereof, for use in the treatment of cancer in a subject, wherein said treatment comprises the separate, sequential or simultaneous administration of i) said selective PARP1 inhibitor, or a pharmaceutically acceptable salt thereof, and ii) a selective estrogen degrader, or a pharmaceutically acceptable salt thereof, to said subject.

In some embodiments, disclosed is a selective estrogen degrader, or a pharmaceutically acceptable salt thereof, for use in the treatment of cancer in a subject, wherein said treatment comprises the separate, sequential or simultaneous administration of i) said selective estrogen degrader, or a pharmaceutically acceptable salt thereof, and ii) a selective PARP1 inhibitor, or a pharmaceutically acceptable salt thereof, to said subject.

In some embodiments, disclosed is the use of a selective PARP1 inhibitor, or a pharmaceutically acceptable salt thereof, in the manufacture of a medicament for use in the treatment of cancer, wherein said treatment comprises the separate, sequential or simultaneous administration of i) said medicament comprising said selective PARP1 inhibitor, or a pharmaceutically acceptable salt thereof, and ii) a selective estrogen degrader, or a pharmaceutically acceptable salt thereof, to said subject.

In some embodiments, disclosed is the use of a selective estrogen degrader, or a pharmaceutically acceptable salt thereof, in the manufacture of a medicament for use in the treatment of cancer, wherein said treatment comprises the separate, sequential or simultaneous administration of i) said medicament comprising said selective estrogen degrader, or a pharmaceutically acceptable salt thereof, and ii) a selective PARP1 inhibitor, or a pharmaceutically acceptable salt thereof, to said subject.

In the above embodiments, the cancer may be breast cancer or breast cancer which is ER positive (ER+).

In some embodiments, disclosed is a pharmaceutical product comprising i) a selective PARP1 inhibitor or a pharmaceutically acceptable salt thereof, and ii) a selective estrogen degrader or a pharmaceutically acceptable salt thereof.

In some embodiments, disclosed is a kit comprising: a first pharmaceutical composition comprising a selective PARP1 inhibitor, or a pharmaceutically acceptable salt thereof; a second pharmaceutical composition comprising a selective estrogen degrader, or a pharmaceutically acceptable salt thereof; and instructions for using the first and second pharmaceutical compositions in combination.

The combination of a selective PARP1 inhibitor and a selective estrogen degrader may result in fewer side effects or be more effective than current monotherapies or combination therapies. This may result from the selective nature of the PARP1 inhibitor and/or the selective nature of the estrogen degrader.

### BRIEF DESCRIPTION OF DRAWINGS

FIG. 1 shows the relative tumour volume of mice over time dosed with various agents in a ST4316B patient derived xenograft (PDX) model.

FIG. 2 shows the relative tumour volume of mice over time dosed with various agents in a CTG-1124 patient derived xenograft (PDX) model.

FIG. 3 shows the relative tumour volume of mice over time dosed with various agents in a PDX583.2 patient derived xenograft (PDX) model.

### DETAILED DESCRIPTION

### Selective PARP1 Inhibitors

Selective PARP1 inhibitors are compounds which inhibit PARP1 selectively over other members of the PARP family including PARP2, PARP3, PARP5a and PARP6. Advantageously, the selective PARP1 inhibitor possesses selectivity for PARP1 over PARP2. In an embodiment, the selective PARP1 inhibitor has 10-fold selectivity for PARP1 over PARP2. In a further embodiment, the selective PARP1 inhibitor has 100-fold selectivity for PARP1 over PARP2. In a further embodiment, the selective PARP1 inhibitor has 500-fold selectivity for PARP1 over PARP2.

In some embodiments, the selective PARP1 inhibitor is a compound disclosed in WO2021/013735A1. These compounds are of Formula (I):

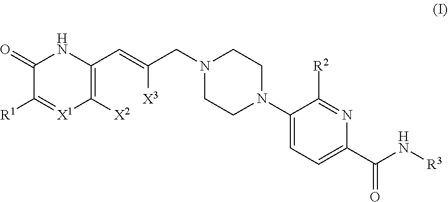

wherein:
X¹ and X² are each independently selected from N and C(H),
X³ is independently selected from N and C(R⁴), wherein R⁴ is H or fluoro,
R¹ is C₁₋₄ alkyl or C₁₋₄ fluoroalkyl,
R² is independently selected from H, halo, C₁₋₄ alkyl, and C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹═C(H), and X³ is C(R⁴), and
when X³ is N, then X¹ and X² are both C(H).

Alkyl groups and moieties are straight or branched chain, e.g. C₁₋₈ alkyl, C₁₋₆ alkyl, C₁₋₄ alkyl or C₅₋₆ alkyl. Examples of alkyl groups are methyl, ethyl, n-propyl, iso-propyl, n-butyl, t-butyl, n-pentyl, n-hexyl, n-heptyl and n-octyl, such as methyl or n-hexyl.

Fluoroalkyl groups are alkyl groups in which one or more H atoms is replaced with one or more fluoro atoms, e.g. C₁₋₈ fluoroalkyl, C₁₋₆ fluoroalkyl, C₁₋₄ fluoroalkyl or C₅₋₆ fluoroalkyl. Examples include fluoromethyl (—CH₂F), difluromethyl (—CHF₂), trifluoromethyl (—CF₃), 2,2,2-trifluoroethyl (CF₃CH₂—), 1,1-difluoroethyl (CH₃CHF₂—), 2,2-difluoroethyl (CHF₂CH₂—), and 2-fluoroethyl (CH₂FCH₂—).

Halo means fluoro, chloro, bromo, and iodo. In an embodiment, halo is fluoro or chloro.

In some embodiments, the selective PARP1 inhibitor is “AZD5305”, which refers to a compound with the chemical name 5-{4-[(7-ethyl-6-oxo-5,6-dihydro-1,5-naphthyridin-3-yl)methyl]piperazin-1-yl}-N-methylpyridine-2-carboxamide and of the structure shown below:

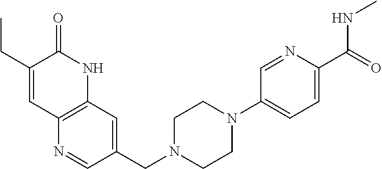 [SMILES: CCc1cc2ncc(CN3CCN(c4ccc(C(=O)NC)nc4)CC3)cc2[nH]c1=O]

AZD5305 is a potent and selective PARP1 inhibitor and PARP1-DNA trapper with excellent in vivo efficacy. AZD5305 is highly selective for PARP1 over other PARP family members, with good secondary pharmacology and physicochemical properties and excellent pharmacokinetics in preclinical species, and with reduced effects on human bone marrow progenitor cells in vitro. The reduced effects on human bone marrow progenitor cells are expected to result in reduced hematological toxicity (such as anemia) compared to non-selective PARP1 inhibitors at clinically relevant doses

The synthesis of AZD5305 is described in Johannes 2021 and in WO2021/013735, the contents of which are hereby incorporated by reference in their entirety. In some embodiments, a free base AZD5305 is administered to a subject. In some embodiments, a pharmaceutically acceptable salt of AZD5305 is administered to a subject. In some embodiments, crystalline AZD5305 or a pharmaceutically acceptable salt of AZD5305 is administered to a subject.

In some embodiments, the selective PARP1 inhibitor is “AZ14114554”, which refers to a compound with the chemical name 7-((4-(1,5-dimethyl-1H-imidazol-2-yl) piperazin-1-yl)methyl)-3-ethylquinolin-2 (1H)-one and the structure shown below:

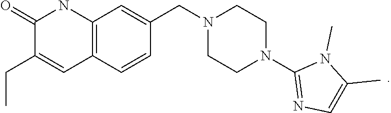 [SMILES: CCc1cc2ccc(CN3CCN(c4ncc(C)n4C)CC3)cc2[nH]c1=O]

The synthesis of AZ14114554 is described in Johannes 2021 (compound 16), the contents of which are hereby incorporated by reference in their entirety. In some embodiments, a free base AZ14114554 is administered to a subject. In some embodiments, a pharmaceutically acceptable salt of AZ14114554 is administered to a subject. In some embodiments, crystalline AZ14114554 or a pharmaceutically acceptable salt of AZ14114554 is administered to a subject.

In some embodiments, the selective PARP1 inhibitor is a compound disclosed in any one of WO2010/133647, WO2011/006794, WO2011/006803, WO2013/014038, WO2013/076090 and WO2014/064149, which are herein incorporated by reference. These selective PARP1 inhibitors have a core which is:

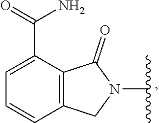 [SMILES: *N1Cc2cccc(C(N)=O)c2C1=O]

and which in some embodiments is:

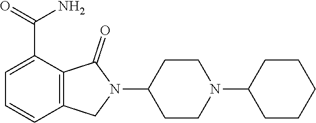 [SMILES: NC(=O)c1cccc2c1C(=O)N(C1CCN(C3CCCCC3)CC1)C2]

Compounds of particular interest are:

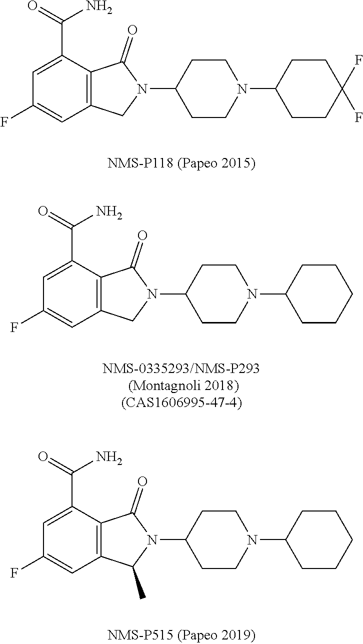 [SMILES: NC(=O)c1cc(F)cc2c1C(=O)N(C1CCN(C3CCC(F)(F)CC3)CC1)C2 ; NC(=O)c1cc(F)cc2c1C(=O)N(C1CCN(C3CCCCC3)CC1)C2 ; C[C@H]1c2cc(F)cc(C(N)=O)c2C(=O)N1C1CCN(C2CCCCC2)CC1]

### Selective Estrogen Degraders

Selective estrogen degraders (SERDs) bind to the estrogen receptor causing it to be degraded and therefore downregulated.

In some embodiments the selective estrogen degrader is a next generation selective estrogen degrader (ngSERD, for example giredestrant or a pharmaceutically acceptable salt thereof, elacestrant or a pharmaceutically acceptable salt thereof, imlunestrant or a pharmaceutically acceptable salt thereof or camizestrant or a pharmaceutically acceptable salt thereof).

In some embodiments the selective estrogen receptor degrader is selected from fulvestrant or a pharmaceutically acceptable salt thereof, giredestrant or a pharmaceutically acceptable salt thereof, elacestrant or a pharmaceutically acceptable salt thereof, imlunestrant or a pharmaceutically acceptable salt thereof and camizestrant or a pharmaceutically acceptable salt thereof.

In embodiments the selective estrogen receptor degrader is selected from fulvestrant or a pharmaceutically acceptable salt thereof, giredestrant or a pharmaceutically acceptable salt thereof and elacestrant or a pharmaceutically acceptable salt thereof.

In some embodiments the selective estrogen receptor degrader is selected from giredestrant or a pharmaceutically acceptable salt thereof, elacestrant or a pharmaceutically acceptable salt thereof, imlunestrant or a pharmaceutically acceptable salt thereof and camizestrant or a pharmaceutically acceptable salt thereof.

In some embodiments the selective estrogen receptor degrader is selected from giredestrant or a pharmaceutically acceptable salt thereof and elacestrant or a pharmaceutically acceptable salt thereof.

In some embodiments the selective estrogen receptor degrader is camizestrant or a pharmaceutically acceptable salt thereof.

In some embodiments the selective estrogen receptor degrader is fulvestrant or a pharmaceutically acceptable salt thereof.

In some embodiments the selective estrogen receptor degrader is giredestrant or a pharmaceutically acceptable salt thereof.

In some embodiments the selective estrogen receptor degrader is imlunestrant or a pharmaceutically acceptable salt thereof.

In some embodiments the selective estrogen receptor degrader is camizestrant or a pharmaceutically acceptable salt thereof.

In some embodiments the selective estrogen receptor degrader is a PROTAC (proteolysis targeting chimera).

In some embodiments the selective estrogen receptor degrader is ARV-471 or a pharmaceutically acceptable salt thereof.

In some embodiments the selective estrogen receptor degrader is administered orally.

The term “camizestrant” (AZD9833) refers to a compound with the chemical name N-[1-(3-fluoropropyl) azetidin-3-yl]-6-[(6S,8R)-8-methyl-7-(2,2,2-trifluoroethyl)-6,7,8,9-tetrahydro-3H-pyrazolo[4,3-]isoquinolin-6-yl]-pyridin-3-amine and structure shown below:

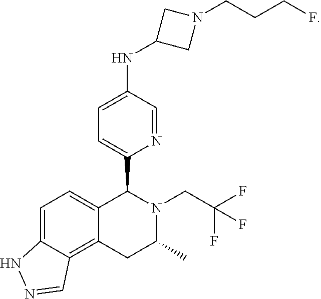

Camizestrant is disclosed in WO2018/077630A1 and Scott 2020, the contents of which are hereby incorporated by reference in its entirety. In some embodiments, a free base camizestrant is administered to a subject. In some embodiments, a pharmaceutically acceptable salt of camizestrant is administered to a subject.

Imlunestrant (LY-3484356) has the following chemical structure:

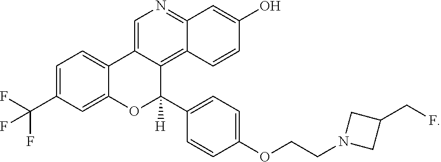 [SMILES: Oc1ccc2c3c(cnc2c1)-c1ccc(C(F)(F)F)cc1O[C@@H]3c1ccc(OCCN2CC(CF)C2)cc1]

The free base of imlunestrant is known by the chemical name (58)-5-[4-[2-[3-(fluoromethyl) azetidin-1-yl]ethoxy]phenyl]-8-(trifluoromethyl)-5H-chromeno[4,3-c]quinolin-2-ol. Imlunestrant is disclosed in WO2020/014435A1.

Giredestrant (GDC-9545) has the following chemical structure:

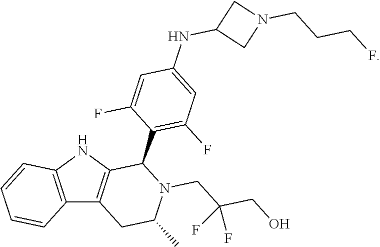 [SMILES: C[C@@H]1Cc2c([nH]c3ccccc23)[C@@H](c2c(F)cc(NC3CN(CCCF)C3)cc2F)N1CC(F)(F)CO]

The free base of giredestrant is known by the chemical name 3-[(1R,3R)-1-[2,6-difluoro-4-[[1-(3-fluoropropyl) azetidin-3-yl]amino]phenyl]-3-methyl-1,3,4,9-tetrahydropyrido[3,4-b]indol-2-yl]-2,2-difluoropropan-1-ol. Giredestrant is disclosed in WO2016/097072A1.

ARV-471 has the following chemical structure:

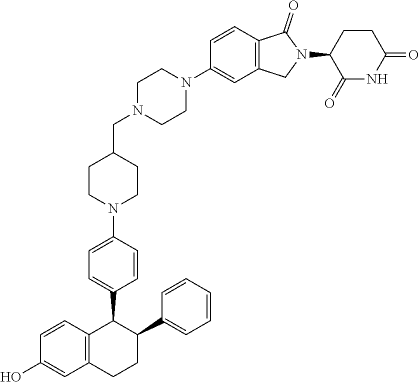 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

ARV-471 is disclosed in WO2018/102725A1.

### Combinations

In some embodiments, the selective PARP1 inhibitor is either AZD5305 or AZD9574 and the SERD is camizestrant. In some of these embodiments, the selective PARP1 inhibitor is AZD5305 and the SERD is camizestrant.

The language “pharmaceutical composition” includes compositions comprising an active ingredient and a pharmaceutically acceptable excipient, carrier or diluent, wherein the active ingredient is a selective PARP1 inhibitor or a pharmaceutically acceptable salt thereof, or a selective estrogen degrader or a pharmaceutically acceptable salt thereof. The language “pharmaceutically acceptable excipient, carrier or diluent” includes compounds, materials, compositions, and/or dosage forms which are, within the scope of sound medical judgment, suitable for use in contact with the tissues of human beings and animals without excessive toxicity, irritation, allergic response, or other problem or complication, as ascertained by one of skill in the art. In some embodiments, the pharmaceutical compositions are in solid dosage forms, such as capsules, tablets, granules, powders or sachets. In some embodiments, the pharmaceutical compositions are in the form of a sterile injectable solution in one or more aqueous or non-aqueous non-toxic parenterally acceptable buffer systems, diluents, solubilizing agents, co-solvents, or carriers. A sterile injectable preparation may also be a sterile injectable aqueous or oily suspension or suspension in a non-aqueous diluent, carrier or co-solvent, which may be formulated according to known procedures using one or more of the appropriate dispersing or wetting agents and suspending agents. The pharmaceutical compositions could be a solution for iv bolus/infusion injection or a lyophilized system (either alone or with excipients) for reconstitution with a buffer system with or without other excipients. The lyophilized freeze-dried material may be prepared from non-aqueous solvents or aqueous solvents. The dosage form could also be a concentrate for further dilution for subsequent infusion.

The language “treat,” “treating” and “treatment” includes the reduction or inhibition of enzyme or protein activity related to PARP-1, ER or cancer in a subject, amelioration of one or more symptoms of cancer in a subject, or the slowing or delaying of progression of cancer in a subject. The language “treat,” “treating” and “treatment” also includes the reduction or inhibition of the growth of a tumor or proliferation of cancerous cells in a subject.

The language “inhibit”, “inhibition” or “inhibiting” includes a decrease in the baseline activity of a biological activity or process.

The term “subject” includes warm-blooded mammals, for example, primates, dogs, cats, rabbits, rats, and mice. In some embodiments, the subject is a primate, for example, a human. In some embodiments, the subject is suffering from cancer.

The language “therapeutically effective amount” includes that amount of a selective PARP1 inhibitor (such as AZD5305) and that amount of a selective estrogen degrader (such as camizestrant) which together will elicit a biological or medical response in a subject, for example, the reduction or inhibition of enzyme or protein activity related to PARP1, ER, or cancer; amelioration of symptoms of cancer; or the slowing or delaying of progression of cancer. In some embodiments, the language “therapeutically effective amount” includes the amount of a selective PARP1 inhibitor (such as AZD5305) and a selective estrogen degrader (such as camizestrant) together that is effective to at least partially alleviate, inhibit, and/or ameliorate cancer, or inhibit PARP1 or ER, and/or reduce or inhibit the growth of a tumor or proliferation of cancerous cells in a subject.

In some embodiments, disclosed is a method of treating cancer in a subject in need thereof, comprising administering to the subject a first amount of AZD5305 or a pharmaceutically acceptable salt thereof, and a second amount of camizestrant or a pharmaceutically acceptable salt thereof. In the method, the first amount and the second amount together comprise a therapeutically effective amount.

In some embodiments, disclosed is a selective PARP1 inhibitor, or a pharmaceutically acceptable salt thereof, for use in the treatment of cancer in a subject, wherein said treatment comprises the separate, sequential or simultaneous administration of i) said selective PARP1 inhibitor, or a pharmaceutically acceptable salt thereof, and ii) a selective estrogen degrader, or a pharmaceutically acceptable salt thereof, to said subject.

In some embodiments, disclosed is a selective estrogen degrader, or a pharmaceutically acceptable salt thereof, for use in the treatment of cancer in a subject, wherein said treatment comprises the separate, sequential or simultaneous administration of i) said selective estrogen degrader, or a pharmaceutically acceptable salt thereof, and ii) a selective PARP1 inhibitor, or a pharmaceutically acceptable salt thereof, to said subject.

In some embodiments, disclosed is the use of a selective PARP1 inhibitor, or a pharmaceutically acceptable salt thereof, in the manufacture of a medicament for use in the treatment of cancer in a subject, wherein said treatment comprises the separate, sequential or simultaneous administration of i) said medicament comprising said selective PARP1 inhibitor, or a pharmaceutically acceptable salt thereof, and ii) a selective estrogen degrader, or a pharmaceutically acceptable salt thereof, to said subject.

In some embodiments, disclosed is the use of a selective estrogen degrader, or a pharmaceutically acceptable salt thereof, in the manufacture of a medicament for use in the treatment of cancer in a subject, wherein said treatment comprises the separate, sequential or simultaneous administration of i) said medicament comprising said selective estrogen degrader, or a pharmaceutically acceptable salt thereof, and ii) selective PARP1 inhibitor, or a pharmaceutically acceptable salt thereof, to said subject.

In some embodiments, a selective PARP1 inhibitor or a pharmaceutically acceptable salt thereof and a selective estrogen degrader or a pharmaceutically acceptable salt thereof are administered separately, sequentially or simultaneously in a treatment cycle. In some embodiments, a selective PARP1 inhibitor or a pharmaceutically acceptable salt thereof is continuously administered in the treatment cycle and a selective estrogen degrader or a pharmaceutically acceptable salt is also continuously administered in the treatment cycle.

The term “continuous” or “continuously” refers to administration of a therapeutic agent, e.g. a selective PARP1 inhibitor, at regular intervals without stopping or interruption, i.e., no void day. By “void day”, it is meant a day when a therapeutic agent is not administered.

A “cycle”, “treatment cycle” or “dosing schedule”, as used herein, refers to a period of combination treatment that is repeated on a regular schedule. For example, the treatment can be given for one week, two weeks, or three weeks wherein the selective PARP1 inhibitor and the selective estrogen degrader are administered in a coordinated fashion. In some embodiments, a treatment cycle is about 1 week to about 3 months. In some embodiments, a treatment cycle is about 5 days to about 1 month. In some embodiments, a treatment cycle is about 1 week to about 3 weeks. In some embodiments, a treatment cycle is about 1 week, about 10 days, about 2 weeks, about 3 weeks, about 4 weeks, about 2 months, or about 3 months.

In some embodiments, a selective PARP1 inhibitor or a pharmaceutically acceptable salt thereof and a selective estrogen degrader or a pharmaceutically acceptable salt thereof are administered to the human subject in one or more treatment cycles, e.g., a treatment course. A “treatment course” comprises multiple treatment cycles, which can be repeated on a regular schedule, or adjusted as a tapered schedule as the patient's disease progression is monitored. For example, a patient's treatment cycles can have longer periods of treatment and/or shorter periods of rest at the beginning of a treatment course (e.g., when the patient is first diagnosed), and as the cancer enters remission, the rest period lengthens, thereby increasing the length of one treatment cycle. The period of time for treatment and rest in a treatment cycle, the number of treatment cycles, and the length of time for the treatment course can be determined and adjusted throughout the treatment course by the skilled artisan based on the patient's disease progression, treatment tolerance, and prognosis. In some embodiments, the method comprises 1 to 10 treatment cycles. In some embodiments, the method comprises 2 to 8 treatment cycles.

### AZD5305 Dosing

In some embodiments, AZD5305 or a pharmaceutically acceptable salt thereof is administered for 7 days in a 7-day pre-treatment cycle, and then 28 days in a 28-day treatment cycle, and camizestrant or a pharmaceutically acceptable salt thereof is administered for 28 days in the 28-day treatment cycle.

In some embodiments, AZD5305 or a pharmaceutically acceptable salt thereof is administered orally. In some embodiments, AZD5305 or a pharmaceutically acceptable salt thereof is in tablet dosage form. In some embodiments, AZD5305 is administered in a dose of up to about 60 mg (for example, up to about 5 mg, up to about 10 mg, up to about 15 mg, up to about 20 mg, up to about 25 mg, up to about 30 mg, up to about 35 mg, up to about 40 mg, up to about 45 mg, up to about 50 mg, up to about 55 mg, or up to about 60 mg

AZD5305) per day. In some embodiments, AZD5305 is administered once a day (QD). In some embodiments, AZD5305 is administered in a dose of about 10 mg QD, about 15 mg QD, about 20 mg QD, about 25 mg QD, about 30 mg QD, about 35 mg QD, about 40 mg QD, about 45 mg QD, about 50 mg QD, about 55 mg QD or about 60 mg QD.

In some further embodiments, AZD5305 is administered in a dose of up to about 140 mg (for example, up to about 80 mg, up to about 90 mg, up to about 100 mg, up to about 110 mg, up to about 120 mg, or up to about 140 mg AZD5305) per day. In some further embodiments, AZD5305 is administered in a dose of about 80 mg QD, about 90 mg QD, about 100 mg QD, about 110 mg QD, about 120 mg QD, or about 140 mg QD.

### PARP1 Selective Inhibitor Dosing

In some embodiments, the PARP1 selective inhibitor may be dosed in the same manner as AZD5305 described above.

### Camizestrant Dosing

In some embodiments, camizestrant or a pharmaceutically acceptable salt thereof is administered orally. In some embodiments, camizestrant or a pharmaceutically acceptable salt thereof is in tablet dosage form. In some embodiments, camizestrant or a pharmaceutically acceptable salt thereof is in capsule dosage form. In some embodiments, camizestrant or a pharmaceutically acceptable salt thereof is administered in a dose of about 50 mg to about 150 mg orally once a day (QD). In some of these embodiments camizestrant or a pharmaceutically acceptable salt thereof is administered in a dose of about 50 mg QD, about 60 mg QD, about 70 mg QD, about 75 mg QD, about 80 mg QD, about 90 mg QD, about 100 mg QD, about 110 mg QD, about 120 mg QD, about 130 mg QD, about 140 mg QD or about 150 mg QD. In some embodiments, camizestrant or a pharmaceutically acceptable salt thereof is administered in a dose of about 75 mg QD. In some embodiments, camizestrant or a pharmaceutically acceptable salt thereof is administered in a dose of about 150 mg QD. In some embodiments, the 75 mg dose comprises three 25 mg tablets, or one 75 mg tablet. In some embodiments, the 150 mg dose comprises six 25 mg tablets, or two 75 mg tablets.

### Selective Estrogen Degrader Dosing

In some embodiments, the selective estrogen degrader may be dosed in the same manner as camizestrant described above.

### Combination Dosing

In some embodiments, AZD5305 and camizestrant are taken together on an empty stomach, with no food two hours before, and one hour after. In other embodiments, AZD5305 is taken on an empty stomach, with no food two hours before, and one hour after, and camizestrant is taken with food at least two hours before or one after hour the administration of AZD5305

In some embodiments, disclosed is a pharmaceutical product comprising i) a selective PARP1 inhibitor or a pharmaceutically acceptable salt thereof, and ii) a selective estrogen degrader or a pharmaceutically acceptable salt thereof. In some embodiments, said selective PARP1 inhibitor or a pharmaceutically acceptable salt thereof, and said selective estrogen degrader or a pharmaceutically acceptable salt thereof are present in a single dosage form. In some embodiments, said selective PARP1 inhibitor or a pharmaceutically acceptable salt thereof, and said selective estrogen degrader or a pharmaceutically acceptable salt thereof are present separate dosage forms.

In some embodiments, disclosed is a kit comprising: a first pharmaceutical composition comprising a selective PARP1 inhibitor, or a pharmaceutically acceptable salt thereof; a second pharmaceutical composition comprising a selective estrogen degrader, or a pharmaceutically acceptable salt thereof; and instructions for using the first and second pharmaceutical compositions in combination.

### Cancer

In some embodiments, the cancer treated is breast cancer, i.e. cancer that initially forms in tissues of the breast. The most common type of breast cancer is ductal carcinoma, which begins in the lining of the milk ducts (thin tubes that carry milk from the lobules of the breast to the nipple). Another type of breast cancer is lobular carcinoma, which begins in the lobules (milk glands) of the breast. The term ‘breast cancer’ as used herein includes metastatic breast cancer (mBC), which is breast cancer that has spread to another part of the body.

In particular, the breast cancer may be ER positive (ER+). These are cancers that express receptors for estrogen, and that can have their growth slowed by treatment with estrogen receptor antagonists. In some embodiments the (metastatic) breast cancer may be ER positive (ER+) metastatic breast cancer.

In particular, the (metastatic) breast cancer may be HER2 negative (HER2−ve), meaning that the cancerous cells do not contain high levels of the protein HER2. HER2 stands for human epidermal growth factor receptor 2. HER2 protein is encoded by the HER2 gene. HER2 proteins are receptors that sit on the surface of breast cells. They usually help control the growth and repair of healthy breast tissue.

In some embodiments, the breast cancer treated may be deficient in Homologous Recombination (HR) dependent DNA DSB repair activity. The HR dependent DNA DSB repair pathway repairs double-strand breaks (DSBs) in DNA via homologous mechanisms to reform a continuous DNA helix (Khanna and Jackson 2001). The components of the HR dependent DNA DSB repair pathway include, but are not limited to, ATM (NM_000051), RAD51 (NM_002875), RAD51L1 (NM_002877), RAD51C(NM_002876), RAD51L3 (NM_002878), DMC1 (NM_007068), XRCC2 (NM_005431), XRCC3 (NM_005432), RAD52 (NM_002879), RAD54L (NM_003579), RAD54B (NM_012415), BRCA1 (NM_007295), BRCA2 (NM_000059), RAD50 (NM_005732), MRE11A (NM_005590) and NBS1 (NM_002485). Other proteins involved in the HR dependent DNA DSB repair pathway include regulatory factors such as EMSY (Hughes-Davies 2003). HR components are also described in Wood 2001.

A breast cancer which is deficient in HR dependent DNA DSB repair may comprise or consist of one or more cancer cells which have a reduced or abrogated ability to repair DNA DSBs through that pathway, relative to normal cells i.e. the activity of the HR dependent DNA DSB repair pathway may be reduced or abolished in the one or more cancer cells.

The activity of one or more components of the HR dependent DNA DSB repair pathway may be abolished in the one or more breast cancer cells of an individual having a breast cancer which is deficient in HR dependent DNA DSB repair. Components of the HR dependent DNA DSB repair pathway are well characterised in the art (see for example, Wood 2001) and include the components listed above.

In some embodiments, the breast cancer cells may have a BRCA1 and/or a BRCA2 deficient phenotype i.e. BRCA1 and/or BRCA2 activity is reduced or abolished in the breast cancer cells. Breast cancer cells with this phenotype may be deficient in BRCA1 and/or BRCA2, i.e. expression and/or activity of BRCA1 and/or BRCA2 may be reduced or abolished in the breast cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies 2003). In some embodiments the breast cancer cells may have a BRCA1 and/or a BRCA2 and/or a PALB2 deficient phenotype.

BRCA1 and BRCA2 are known tumour suppressors whose wild-type alleles are frequently lost in tumours of heterozygous carriers (Jasin 2002; Tutt 2002).

In some embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen and Ostrander 1992; Chappuis and Foulkes 2002; Janatová 2003; Jancarkova 2003). Determination of amplification of the BRCA2 binding factor EMSY is described in Hughes-Davies 2003.

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

In some embodiments, the cancer, such as breast cancer or ER+ breast cancer may be BRCA1/2m- or HR-deficient.

In other embodiments, the cancer, such as breast cancer or ER+ breast cancer may have wild-type BRCA1/2.

In some embodiments, the cancer, such as breast cancer or ER+ breast cancer may express PARP1 mRNA.

In some embodiments, the cancer, such as breast cancer or ER+ breast cancer may be HER2 negative.

In some embodiments, the cancer, such as breast cancer or ER+ breast cancer may be tamoxifen resistant.

In some embodiments, the cancer, such as breast cancer or ER+ breast cancer may be resistant to CDK4/6 inhibitors, such as palbociclib, ribociclib, or abemaciclib.

In some embodiments, the cancer, such as breast cancer or ER+ breast cancer has an ESR1 mutation.

In some embodiments, the cancer is metastatic breast cancer.

In some embodiments, the cancer is ER+ve metastatic breast cancer.

In some embodiments, the cancer is ER+ve, HER2−ve metastatic breast cancer. In some embodiments, the cancer is ER+ve, BRCA1/2 and/or PALB2 mutated HER2−ve metastatic breast cancer.

In some embodiments, the breast cancer is BRCA1m (BRCA1 mutated), BRCA2m (BRCA2 mutated), PALB2m (PALB2 mutated), RAD51Cm (RAD51C mutated), or RAD51Dm (RAD51D mutated) HER2-negative breast cancer.

In some embodiments, the breast cancer may be ER-positive HER2-negative breast cancer. “ER-positive HER2-negative breast cancer” comprises tumors with estrogen receptors (are ER-positive) that do not have high levels of the HER2 gene or the HER2 protein (are HER2-negative). ER-positive and HER2-negative status can be determined by methods known in the art, including the use of commercial kits.

In some embodiments, the breast cancer may be ER-positive HER2-negative advanced breast cancer. In some embodiments, the breast cancer may be HER2-negative advanced breast cancer.

In some embodiments, the breast cancer is in a patient with mutated ESR1 (ESR1m).

Without wishing to be bound by theory, the combination of AZD5305 and camizestrant may be beneficial as PARP1 has been linked to regulation of estrogen dependent transcription and inhibition of PARP1 can inhibit estrogen-dependent growth of ER positive breast cancer cells (Gadad 2021). ER positive breast cancer cells that had acquired resistance to hormone-therapy have been found to be sensitive to olaparib, a PARP1/2 inhibitor (Suzuki 2022). As a result, AZD5305 may sensitize ER positive breast cancer cells to camizestrant.

### EXAMPLES

The compounds of the application will now be further explained by reference to the following non-limiting examples.

### Example 1. Efficacy of AZD5305 Combined with ER-Targeting Agents in an In Vivo Pre-Clinical Model ST4316B

ST4316B patient derived xenograft (PDX) model was engrafted subcutaneously (approximately 70 mg fragment) onto the flank of study mice (female Athymic Nude mice, aged 6-12 weeks). When tumours reached approximately 200 mm³, 70 mice with the most similar sized tumours were randomly assigned to treatment groups as demonstrated in the table below. ST4316B is an ER+ and BRCA2m model.

| Group | n | Treatment | Dose | Schedule |
|---|---|---|---|---|
| 1 | 10 | Vehicle (AZD5305) + | | QD + |
| | | Vehicle (camizestrant)^{&} | | QD |
| 2 | 10 | AZD5305 | 1 mg/kg | QD |
| 3 | 10 | Camizestrant | 10 mg/kg | QD |
| 4 | 10 | AZD5305 + | 1 mg/kg + | QD + |
| | | camizestrant^{&} | 10 mg/kg | QD |
| 5 | 10 | Fulvestrant | 5 mg/mouse | QW |
| 6 | 10 | AZD5305 + | 1 mg/kg + | QD + |
| | | Fulvestrant^{&} | 5 mg/mouse | QW |
| 7 | 5 | Palbociclib | 50 mg/kg | QD |
| 8 | 5 | Palbociclib + | 50 mg/kg + | QD + |
| | | camizestrant\hat{ } | 10 mg/kg | QD |

^{&}The second agent administered 1 h post the first agent
\hat{ }Administered without a gap

### Dosing Formulations

| | Formulation | Concentration |
|---|---|---|
| AZD5305 | sterile deionized water/HCI pH 3.5-4 | 0.1 mg/ml |
| Camizestrant | 40% TEG (tetraethylene glycol) / | 1 mg/ml |
| | 60% (of 12.5%) Captisol | |
| Fulvestrant | 100% peanut oil | 50 mg/ml |
| Palbociclib | 1% Tween80, pH 4.4-4.6 | 5 mg/ml |

### Study

The mice were dosed for 28 days, with the dose calculated for individual animals on day of dosing, and with a 10 mg/kg dosing volume. Fulvestrant was dosed subcutaneously once weekly. AZD5305, camizestrant and palbociclib were dosed orally once daily. After 28 days, AZD5305 monotherapy, AZD5305+camizestrant, and AZD5305+Fulvestrant groups were further observed for tumour volume changes in the off-treatment period (day 29-165)

### Tumour Measurement

Tumours were measured 2 times per week using digital calipers. The length and width of the tumour were measured and volume calculated using the following formula:

$$volume = ( length \times {width}^{2} ) \times 0.52$$

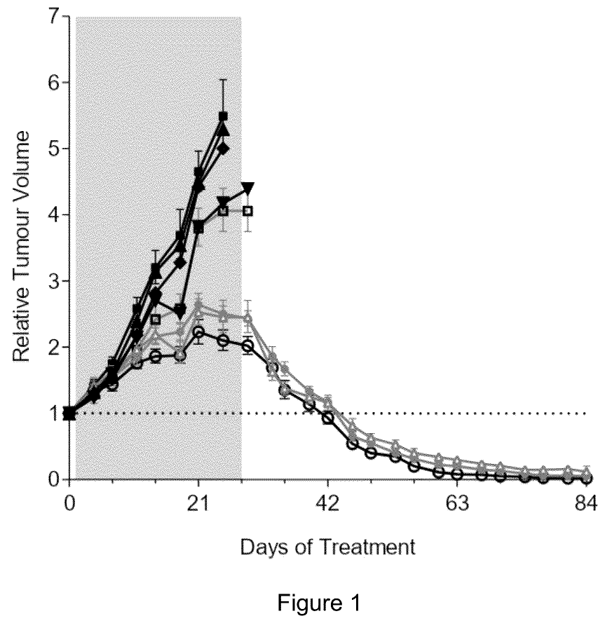

The relative tumour volume is shown in FIG. 1 in which:

| Group | Treatment | Symbol |
|---|---|---|
| 1 | Vehicle (AZD5305) + Vehicle (camizestrant) | [symbol] |
| 2 | AZD5305 | [symbol] |
| 3 | Camizestrant | [symbol] |
| 4 | AZD5305 + camizestrant | [symbol] |
| 5 | Fulvestrant | [symbol] |
| 6 | AZD5305 + Fulvestrant | [symbol] |
| 7 | Palbociclib | [symbol] |
| 8 | Palbociclib + camizestrant | [symbol] |

### Bodyweight

The bodyweight of all mice in the study was measured and recorded 2 times per week; this information was used to calculate precise dosing for each animal.

### Example 2. Efficacy of AZD5305 Combined with ER-Targeting Agents in an In Vivo Pre-Clinical Model CTG-1124

CTG-1124 patient derived xenograft (PDX) model was engrafted subcutaneously onto the flank of study mice (female Athymic Nude mice, aged at least 6-8 weeks). When tumours reached approximately 150-300 mm³, mice with the most similar sized tumours were randomly assigned to treatment groups as demonstrated in the table below. CTG-1124 is an ER+ and BRCA1m model.

| Group | n | Treatment | Dose | Schedule |
|---|---|---|---|---|
| 1 | 10 | Vehicle (AZD5305) + | — | QD + |
| | | Vehicle (camizestrant)^{&} | | QD |
| 2 | 10 | AZD5305 | 1 mg/kg | QD |
| 3 | 10 | Camizestrant | 10 mg/kg | QD |
| 4 | 10 | AZD5305 + | 1 mg/kg + | QD + |
| | | camizestrant^{&} | 10 mg/kg | QD |
| 5 | 10 | Fulvestrant | 5 mg/mouse | QW |
| 6 | 10 | AZD5305 + | 1 mg/kg + | QD + |
| | | Fulvestrant^{&} | 5 mg/mouse | QW |
| 7 | 5 | Palbociclib | 50 mg/kg | QD |
| 8 | 5 | Palbociclib + | 50 mg/kg + | QD + |
| | | camizestrant\hat{ } | 10 mg/kg | QD |

^{&}The second agent administered 1 h post the first agent
\hat{ }Administered without a gap

### Dosing Formulations

| | Formulation | Concentration |
|---|---|---|
| AZD5305 | sterile deionized water/HCI pH 3.5-4 | 0.1 mg/ml |
| Camizestrant | 40% TEG (tetraethylene glycol) / | 1 mg/ml |
| | 60% (of 12.5%) Captisol | |
| Fulvestrant | 100% peanut oil | 250 mg/ml |
| Palbociclib | 1% Tween80, pH 4.4-4.6 | 5 mg/ml |

### Study

The mice were dosed for 35 days, with the dose calculated for individual animals on day of dosing, and with a 10 mg/kg dosing volume. Fulvestrant was dosed subcutaneously once weekly at fixed volume of 0.2 ml per mouse. AZD5305, camizestrant and palbociclib were dosed orally once daily. After 35 days, AZD5305 monotherapy, AZD5305+camizestrant, and AZD5305+Fulvestrant groups were further observed for tumour volume changes in the off-treatment period (day 36-69).

### Tumour Measurement

Tumours were measured 2 times per week using digital calipers. The length and width of the tumour were measured and volume calculated using the following formula:

$$volume = ( length \times {width}^{2} ) \times 0.52$$

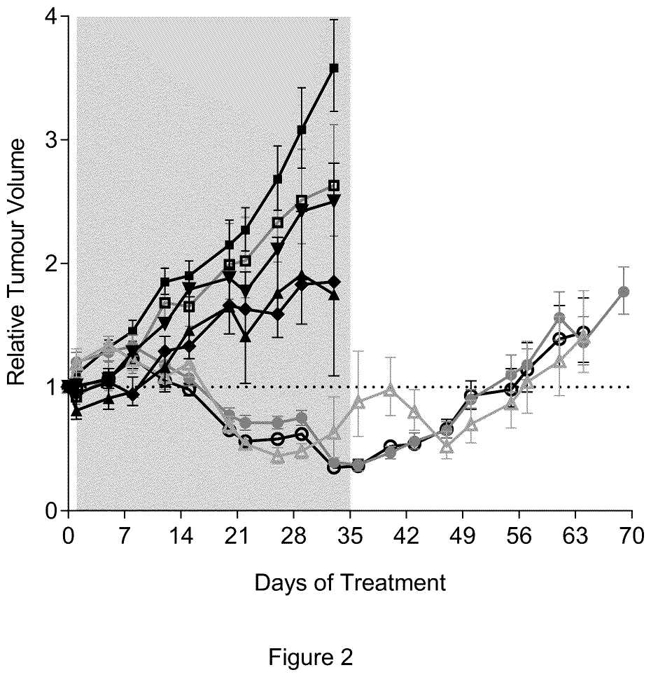

The relative tumour volume is shown in FIG. 2 in which:

| Group | Treatment | Symbol |
|---|---|---|
| 1 | Vehicle (AZD5305) + Vehicle (camizestrant) | [symbol] |
| 2 | AZD5305 | [symbol] |
| 3 | Camizestrant | [symbol] |
| 4 | AZD5305 + camizestrant | [symbol] |
| 5 | Fulvestrant | [symbol] |
| 6 | AZD5305 + Fulvestrant | [symbol] |
| 7 | Palbociclib | [symbol] |
| 8 | Palbociclib + camizestrant | [symbol] |

### Bodyweight

The bodyweight of all mice in the study was measured and recorded 2 times per week; this information was used to calculate precise dosing for each animal.

### Example 3. Efficacy of AZD5305 Combined with ER-Targeting Agents in an In Vivo Pre-Clinical Model PDX583.2

PDX583.2 patient derived xenograft (PDX) model was engrafted subcutaneously onto the flank of study mice (female Athymic Nude mice, aged at least 6-8 weeks). When tumours reached approximately 150-200 mm³, mice were randomly assigned to treatment groups as demonstrated in the table below. PDX583.2 is an ER+ and BRCA2m model.

| Group | n | Treatment | Dose | Schedule |
|---|---|---|---|---|
| 1 | 10 | Untreated | — | — |
| 2 | 10 | AZD5305 | 1 mg/kg | QD |
| 3 | 10 | Camizestrant | 10 mg/kg | QD |
| 4 | 10 | AZD5305 + | 1 mg/kg + | QD + |
| | | camizestrant | 10 mg/kg | QD |
| 5 | 10 | Fulvestrant | 5 mg/mouse | QW |
| 6 | 10 | AZD5305 + | 1 mg/kg + | QD + |
| | | Fulvestrant | 5 mg/mouse | QW |

### Dosing Formulations

| | Formulation | Concentration |
|---|---|---|
| AZD5305 | sterile deionized water/HCI pH 3.5-4 | 0.1 mg/ml |
| Camizestrant | 40% TEG (tetraethylene glycol) / | 1 mg/ml |
| | 60% (of 12.5%) Captisol | |
| Fulvestrant | 100% peanut oil | 50 mg/ml |

### Study

The mice were dosed throughout the study, with the dose calculated for individual animals on day of dosing. Fulvestrant was dosed subcutaneously once weekly at fixed volume of 0.1 ml per mouse. AZD5305 and camizestrant were dosed orally once daily.

### Tumour Measurement

Tumours were measured 2 times per week using digital calipers. The length and width of the tumour were measured and volume calculated using the following formula:

$$volume = ( length \times {width}^{2} ) \times 0.52$$

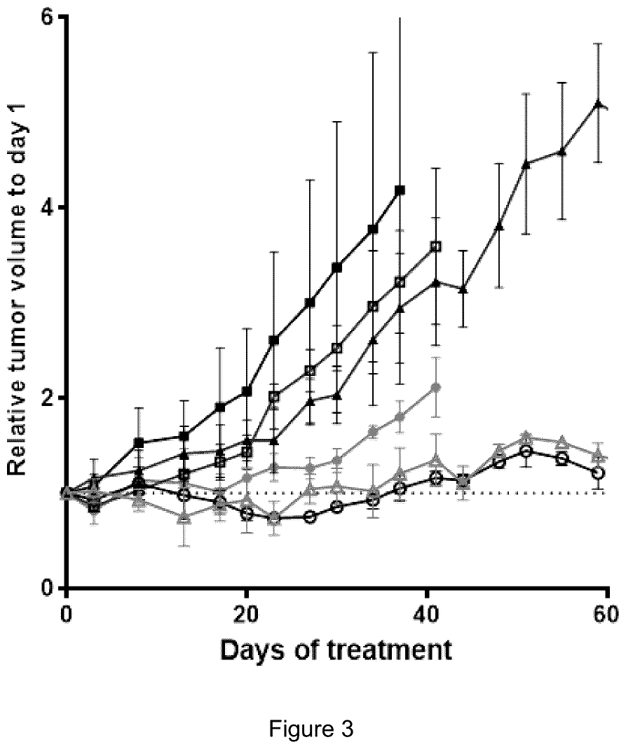

The relative tumour volume is shown in FIG. 3 in which:

| Group | Treatment | Symbol |
|---|---|---|
| 1 | Vehicle (AZD5305) + Vehicle (camizestrant) | [symbol] |
| 2 | AZD5305 | [symbol] |
| 3 | Camizestrant | [symbol] |
| 4 | AZD5305 + camizestrant | [symbol] |
| 5 | Fulvestrant | [symbol] |
| 6 | AZD5305 + Fulvestrant | [symbol] |

### Bodyweight

The bodyweight of all mice in the study was measured and recorded 2 times per week; this information was used to calculate precise dosing for each animal.

### Example 4-Clinical Study of Combination of AZD5305 and Camizestrant to Treat ER-Positive Breast Cancer

In this study, a Cycle 0 monotherapy treatment with AZD5305 precedes the Cycle 1 combination treatment period.

Cycle 0 will be of 7 days duration, and participants receive a single dose of AZD5305 only on Day 1 followed by 6 days of washout where participants received no treatment. Cycle 1 will start the day after completion of Cycle 0.

Camizestrant was dosed at 75 mg QD (once daily) and AZD5305 was dosed at 60 mg QD.

The primary aim of the study was to assess the safety and tolerability of the combination in patients with advanced malignancies, by reviewing the incidence of adverse events and dose-limiting toxicities (DLTs).

A DLT is defined as any toxicity during the periods of Cycle 0 and Cycle 1 (i.e. from dosing on Cycle 0 Day 1 until the last day of dosing in Cycle 1), which includes:
1. Haematological toxicities as follows:
   Grade 4 neutropenia (ANC (Absolute neutrophil count)<500 cells/mm³) lasting longer than 4 consecutive days
   Grade 3 neutropenia (ANC ≥500 to <1000 cells/mm³) of any duration accompanied by fever ≥38.5° C. and/or systemic infection
   Grade 4 thrombocytopenia (<25,000/mm³) lasting >1 day
   Grade 3 thrombocytopenia (25,000 to <50,000/mm³) with clinically significant bleeding attributable to thrombocytopenia
   Any other confirmed haematological toxicity ≥CTCAE (Common Terminology Criteria for Adverse Events) version 5 Grade 4 (a repeat may be required for confirmation of an isolated abnormality in the absence of clinical signs, symptoms or other abnormal investigations, ie, a suspected spurious value)
2. Cardiac DLTs including:
   Symptomatic tachycardia or tachycardia with resting supine heart rate of >125 beats per minute persisting for at least 10 minutes
   Hypotension requiring medical intervention eg, IV fluids.
   Any other cardiac toxicity of CTCAE >Grade 2.
   QTcF (QT interval corrected for heart rate using Fridericia's formula) interval value <340 milliseconds confirmed on at least 2 separate ECGs (Electrocardiograms), recorded 5 minutes apart
   QTcF prolongation >500 milliseconds or QTcF prolongation from baseline by >60 milliseconds confirmed on at least 2 separate ECGs, recorded 5 minutes apart
3. Non-haematological toxicity ≥CTCAE version 5 Grade 3 including:
   Nausea, vomiting or diarrhoea lasting for >72 hours despite administration of maximal supportive therapy
4. Other DLTs:
   Any treatment-related event, including significant dose reductions or omissions, judged to be a DLT. Examples may include confirmed laboratory abnormalities (CTCAE Grade ≥3), CTCAE Grade 2 toxicities that are clinically significant and/or unacceptable according to the investigator, toxicities that result in an inability to administer at least 75% of study treatment during Cycle 1 or delay the administration of study treatment in the subsequent cycle by >7 consecutive days.
   Any death not clearly due to the underlying disease or extraneous causes.

An adverse event (AE) is the development of any untoward medical occurrence in a patient or clinical study patient administered a medicinal product and which does not necessarily have a causal relationship with this treatment. An AE can therefore be any unfavorable and unintended sign (e.g., an abnormal laboratory finding), symptom (for example nausea, chest pain), or disease temporally associated with the use of a medicinal product, whether or not considered related to the medicinal product.

The term AE is used to include both serious and non-serious AEs and can include a deterioration of a pre-existing medical occurrence. An AE may occur at any time, including run-in or washout periods, even if no study treatment has been administered.

A serious adverse event (SAE) is an AE occurring during any study phase (i.e., run-in, treatment, washout, follow-up), that fulfils one or more of the following criteria:
Results in death
Is immediately life threatening
Requires in-patient hospitalisation or prolongation of existing hospitalisation
Results in persistent or significant disability or incapacity
Is a congenital anomaly or birth defect
Is an important medical event that may jeopardise the patient or may require medical treatment to prevent one of the outcomes listed above

‘Life-threatening’ means that the patient was at immediate risk of death from the AE as it occurred, or it is suspected that use or continued use of the product would result in the patient's death. ‘Life-threatening’ does not mean that had an AE occurred in a more severe form it might have caused death (e.g., hepatitis that resolved without hepatic failure).

Outpatient treatment in an emergency room is not in itself a serious AE, although the reasons for it may be (e.g., bronchospasm, laryngeal oedema). Hospital admissions and/or surgical operations planned before or during a study are not considered AEs if the illness or disease existed before the patient was enrolled in the study, provided that it did not deteriorate in an unexpected way during the study.

The grading scales found in the revised NCI CTCAE v5.0 were utilized for all events with an assigned CTCAE grading. For those events without assigned CTCAE grades, the recommendation in the CTCAE criteria that converts mild, moderate and severe events into CTCAE grades was used.

For the 13 patients treated, the summary of the safety data is presented below (at the data cutoff):

| | Number |
|---|---|
| | (percentage) |
| DLTs | 0 |
| Grade 3+ AEs | 4 (30.7%) |
| Grade 3+ AEs related to Camizestrant and AZD5305 | 4 (30.7%) |
| SAEs | 3 (23%) |
| AEs leading to discontinuation | 1 (7.7%) |
| AEs leading to interruption | 6 (46.1%) |
| AE of special interest: Eye disorders² | 5 (38.5%) |
| AE of special interest: Sinus Bradycardia | 1 (7.7%) |

¹ Reported terms: Anaemia/back pain/hip pain (same patient), Neutrophil count decrease, nausea/vomiting, and renal colic/anaemia.
²Eye disorders: all reported as gr 1. Reported terms: Eye disorders, Flashing lights, Photopsia/Dry eye/ Photophobia, Visual disturbance, Cotton wool spots.

Of these 13 patients, one showed a partial response at the 8 week scan, and 5 shows stable disease at the 8 week scan. A partial response is defined as at least a 30% decrease in the sum of the diameters of target lesions, taking as reference the baseline sum diameters. Progressive disease is defined as at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. Stable disease is defined as neither sufficient shrinkage to qualify for partial response nor sufficient increase to qualify for progressive disease, taking as reference the smallest sum of diameters while on study.

### Example 5

A phase 3 trial entitled “Saruparib (AZD5305) plus Camizestrant compared with CDK4/6 Inhibitor Plus Endocrine Therapy or Plus Camizestrant in HR-Positive, HER2-Negative (IHC 0, 1+, 2+/ISH non-amplified), BRCA1, BRCA2, or PALB2m Advanced Breast Cancer-EvoPAR-BR01” with study identifier D9722C00001 has started on 18 Apr. 2024. In the experimental arm, patients will receive saruparib (AZD5305) orally plus camizestrant orally.

### REFERENCES

A number of publications are cited above in order to more fully describe and disclose the invention and the state of the art to which the invention pertains. Full citations for these references are provided below. The entirety of each of these references is incorporated herein.

| | Reference | Doi |
|---|---|---|
| Alisson 2020 | Allison KH, et al, J Clin | 10.1200/JCO.19.02309 |
| | Oncol. (2020) 38(12), 1346-1366 | |
| Cardoso 2017 | Cardoso F, et al. Ann Oncol. | 10.1093/annonc/mdw544 |
| | (2017) 28, 13-33 | |
| Chappuis and | Chappuis PO and Foulkes WD, | 10.1007/978-1-4757-3587-1_2 |
| Foulkes 2002 | Cancer Treat Res, 107, 29-59 | |
| | (2002) | |
| Chen 2022 | Chen, YC, et al, Expert Opinion on | 10.1080/13543784.2021.1983542 |
| | Investigational Drugs (2022) | |
| | 31(6), 515-529 | |
| denBrok 2017 | denBrok WD, et al. JCO Precis | 10.1200/PO.16.00031 |
| | Oncol. (2017) 1: 1-13 | |
| Gadad 2021 | Gadad SS, et al, Mol Cancer Res. | 10.1158/1541-7786.MCR-21-0103 |
| | (2021) 19(10), 1688-1698 | |
| Harvey 1999 | Harvey JM, et al, J Clin | 10.1200/JCO.1999.17.5.1474 |
| | Oncol. (1999) 17(5), 1474-1481 | |
| Hughes-Davies | Hughes-Davies, et al., Cell, 115, | 10.1016/s0092-8674(03)00930-9 |
| 2003 | 523-535 (2003) | |
| Janatova 2003 | Janatova M, et al., Neoplasma, | PMID: 12937835 |
| | 50(4), 246-50 (2003) | |
| Jancárková | Jancárková N, Ceska Gynekol., | PMID: 12708108 |
| 2003 | 68(1), 11-6 (2003) | |
| Jasin 2002 | Jasin M, Oncogene, 21(58), 8981- | 10.1038/sj.onc.1206176 |
| | 93 (2002) | |
| Johannes 2021 | Johannes JW, et al., J Med Chem | 10.1021/acs.jmedchem.1c01012 |
| | 2021, 64, 14498-14512 | |
| Khanna and | Khanna KK and Jackson SP, Nat. | 10.1038/85798 |
| Jackson 2001 | Genet. 27(3): 247-254 (2001) | |
| Lumachi 2015 | Lumachi F, et al, World J Biol | 10.4331/wjbc.v6.i3.231 |
| | Chem. (2015) 6(3), 231-239 | |
| Lüönd 2021 | Lüönd F, et al, Br J Cancer (2021) | 10.1038/s41416-021-01328-7 |
| | 125(2), 164-175 | |
| Neuhausen and | Neuhausen SL and Ostrander EA, | 10.1089/gte.1997.1.75 |
| Ostrander 1992 | Genet. Test, 1, 75-83 (1992) | |
| Robertson 2007 | Robertson JF, Oncologist (2007), | 10.1634/theoncologist.12-7-774 |
| | 12, 774-784 | |
| Scott 2020 | Scott, JS, et al, J Med Chem | 10.1021/acs.jmedchem.0c01163 |
| | (2020), 63, 23, 14530-14559 | |
| Sharma 2019 | Sharma R, Breast Cancer (2019) | 10.1007/s12282-018-00941-4 |
| | 26(4), 428-445 | |
| Stewart 2022 | Stewart MD, et al, Oncologist. | 10.1093/oncolo/oyab053 |
| | (2022) 27(3), 167-174 | |
| Stravodimou | Stravodimou A, et al, Anticancer | 10.21873/anticanres.14486 |
| 2020 | Res. 2020; 40(9): 4829-4841 | |
| Sung 2021 | Sung H, et al, Ca Cancer J | 10.3322/caac.21660 |
| | Clin. (2021) 71(3), 209-249 | |
| Suzuki 2022 | Suzuki Y, et al, Breast Cancer | 10.1007/s12282-021-01282-5 |
| | (2022) 29(1), 77-91 | |
| Tutt 2002 | Tutt, et al., Trends Mol Med., | 10.1016/s1471-4914(02)02434-6 |
| | 8(12), 571-576, (2002) | |
| Wood 2001 | Wood, et al., Science, 291, 1284- | 10.1126/science.1056154 |
| | 1289 (2001) | |

CTCAE v5.0 is available from the CTEP website.

### NUMBERED STATEMENTS

1. A method of treating cancer in a subject in need thereof, comprising administering to the subject a first amount of a selective PARP1 inhibitor or a pharmaceutically acceptable salt thereof, and a second amount of a selective estrogen degrader or a pharmaceutically acceptable salt thereof, wherein the first amount and the second amount together comprise a therapeutically effective amount.

2. The method according to statement 1, wherein the cancer is:
a) breast cancer;
b) ER+ breast cancer;
c) ER+ breast cancer which is BRCA1/2m; or
d) ER+ breast cancer which is HR deficient.

3. The method according to either statement 1 or statement 2, wherein the selective PARP inhibitor is AZD5305.

4. The method according to any one of statements 1 to 3, wherein the selective estrogen degrader is camizestrant.

5. The method according to either statement 3 or statement 4 wherein AZD5305 is administered once daily.

6. The method according to statement 5, wherein AZD5305 is administered in a dose of up to about 60 mg per day.

7. The method according to statement 6, wherein AZD5305 is administered in a dose of 60 mg per day.

8. The method according to statement 6, wherein AZD5305 is administered in a dose of 20 mg per day.

9. The method according to any one of statements 4 to 8, wherein the selective estrogen degrader is camizestrant, and is administered once daily.

10. The method according to statement 9, wherein camizestrant is administered in a dose of about 50 mg to about 150 mg once a day.

11. The method according to statement 10, wherein camizestrant is administered in a dose of 75 mg once a day.

12. A selective PARP1 inhibitor, or a pharmaceutically acceptable salt thereof, for use in the treatment of cancer in a subject, wherein said treatment comprises the separate, sequential or simultaneous administration of i) said selective PARP1 inhibitor, or a pharmaceutically acceptable salt thereof, and ii) a selective estrogen degrader, or a pharmaceutically acceptable salt thereof, to said subject.

13. The selective PARP1 inhibitor, or a pharmaceutically acceptable salt thereof, for use according to statement 12, wherein the cancer is:
a) breast cancer;
b) ER+ breast cancer;
c) ER+ breast cancer which is BRCA1/2m; or
d) ER+ breast cancer which is HR deficient.

14. The selective PARP1 inhibitor, or a pharmaceutically acceptable salt thereof, for use according to either statement 12 or statement 13, wherein the selective PARP inhibitor is AZD5305.

15. The selective PARP1 inhibitor, or a pharmaceutically acceptable salt thereof, for use according to any one of statements 12 to 14, wherein the selective estrogen degrader is camizestrant.

16. The selective PARP1 inhibitor, or a pharmaceutically acceptable salt thereof, for use according to either statement 14 or statement 15, wherein AZD5305 is administered once daily.

17. The selective PARP1 inhibitor, or a pharmaceutically acceptable salt thereof, for use according to statement 16, wherein AZD5305 is administered in a dose of up to about 60 mg per day.

18. The selective PARP1 inhibitor, or a pharmaceutically acceptable salt thereof, for use according to statement 17, wherein AZD5305 is administered in a dose of 60 mg per day.

19. The selective PARP1 inhibitor, or a pharmaceutically acceptable salt thereof, for use according to statement 17, wherein AZD5305 is administered in a dose of 20 mg per day.

20. The selective PARP1 inhibitor, or a pharmaceutically acceptable salt thereof, for use according to any one of statements 15 to 19, wherein the selective estrogen degrader is camizestrant and is administered once daily.

21. The selective PARP1 inhibitor, or a pharmaceutically acceptable salt thereof, for use according to statement 20, wherein camizestrant is administered in a dose of about 50 mg to about 150 mg once a day.

22. The selective PARP1 inhibitor, or a pharmaceutically acceptable salt thereof, for use according to statement 21, wherein camizestrant is administered in a dose of 75 mg once a day.

23. A selective estrogen degrader, or a pharmaceutically acceptable salt thereof, for use in the treatment of cancer in a subject, wherein said treatment comprises the separate, sequential or simultaneous administration of i) said selective estrogen degrader, or a pharmaceutically acceptable salt thereof, and ii) a selective PARP1 inhibitor, or a pharmaceutically acceptable salt thereof, to said subject.

24. The selective estrogen degrader, or a pharmaceutically acceptable salt thereof, for use according to statement 23, wherein the cancer is:
a) breast cancer;
b) ER+ breast cancer;
c) ER+ breast cancer which is BRCA1/2m; or
d) ER+ breast cancer which is HR deficient.

25. The selective estrogen degrader, or a pharmaceutically acceptable salt thereof, for use according to either statement 23 or statement 24, wherein the selective PARP inhibitor is AZD5305.

26. The selective estrogen degrader, or a pharmaceutically acceptable salt thereof, for use according to any one of statements 23 to 25, wherein the selective estrogen degrader is camizestrant.

27. The selective estrogen degrader, or a pharmaceutically acceptable salt thereof, for use according to either statement 25 or statement 26, wherein AZD5305 is administered once daily.

28. The selective estrogen degrader, or a pharmaceutically acceptable salt thereof, for use according to statement 27, wherein AZD5305 is administered in a dose of up to about 60 mg per day.

29. The selective estrogen degrader, or a pharmaceutically acceptable salt thereof, for use according to statement 28, wherein AZD5305 is administered in a dose of 60 mg per day.

30. The selective estrogen degrader, or a pharmaceutically acceptable salt thereof, for use according to statement 28, wherein AZD5305 is administered in a dose of 20 mg per day.

31. The selective estrogen degrader, or a pharmaceutically acceptable salt thereof, for use according to any one of statements 26 to 30, wherein the selective estrogen degrader is camizestrant and is administered once daily.

32. The selective estrogen degrader, or a pharmaceutically acceptable salt thereof, for use according to statement 31, wherein camizestrant is administered in a dose of about 50 mg to about 150 mg once a day.

33. The selective estrogen degrader, or a pharmaceutically acceptable salt thereof, for use according to statement 32, wherein camizestrant is administered in a dose of 75 mg once a day.

34. The use of a selective PARP1 inhibitor, or a pharmaceutically acceptable salt thereof, in the manufacture of a medicament for use in the treatment of cancer wherein said treatment comprises the separate, sequential or simultaneous administration of i) said medicament comprising the selective PARP1 inhibitor, or a pharmaceutically acceptable salt thereof, and ii) a selective estrogen degrader, or a pharmaceutically acceptable salt thereof, to said subject.

35. The use of a selective PARP1 inhibitor, or a pharmaceutically acceptable salt thereof, according to statement 34, wherein the cancer is:
a) breast cancer;
b) ER+ breast cancer;
c) ER+ breast cancer which is BRCA1/2m; or
d) ER+ breast cancer which is HR deficient.

36. The use of a selective PARP1 inhibitor, or a pharmaceutically acceptable salt thereof, according to either statement 34 or statement 35, wherein the selective PARP inhibitor is AZD5305.

37. The use of a selective PARP1 inhibitor, or a pharmaceutically acceptable salt thereof, according to any one of statements 34 to 36, wherein the selective estrogen degrader is camizestrant.

38. The use of a selective PARP1 inhibitor, or a pharmaceutically acceptable salt thereof, according to either statement 36 or statement 37, wherein AZD5305 is administered once daily.

39. The use of a selective PARP1 inhibitor, or a pharmaceutically acceptable salt thereof, according to statement 38, wherein AZD5305 is administered in a dose of up to about 60 mg per day.

40. The use of a selective PARP1 inhibitor, or a pharmaceutically acceptable salt thereof, according to statement 39, wherein AZD5305 is administered in a dose of 60 mg per day.

41. The use of a selective PARP1 inhibitor, or a pharmaceutically acceptable salt thereof, according to statement 39, wherein AZD5305 is administered in a dose of 20 mg per day.

42. The use of a selective PARP1 inhibitor, or a pharmaceutically acceptable salt thereof, according to any one of statements 37 to 41, wherein the selective estrogen degrader is camizestrant and is administered twice daily.

43. The use of a selective PARP1 inhibitor, or a pharmaceutically acceptable salt thereof, according to statement 42, wherein camizestrant is administered in a dose of about 50 mg to about 150 mg once a day.

44. The use of a selective PARP1 inhibitor, or a pharmaceutically acceptable salt thereof, according to statement 43, wherein camizestrant is administered in a dose of 75 mg once a day.

45. A pharmaceutical product comprising i) a selective PARP1 inhibitor or a pharmaceutically acceptable salt thereof, and ii) a selective estrogen degrader or a pharmaceutically acceptable salt thereof.

46. The pharmaceutical product according to statement 45, wherein the selective PARP1 inhibitor is AZD5305.

47. The pharmaceutical product according to either statement 45 or 46, wherein the selective estrogen degrader is camizestrant.

48. A kit comprising: a first pharmaceutical composition comprising a selective PARP1 inhibitor, or a pharmaceutically acceptable salt thereof; a second pharmaceutical composition comprising a selective estrogen degrader, or a pharmaceutically acceptable salt thereof; and instructions for using the first and second pharmaceutical compositions in combination.

49. The kit according to statement 48, wherein the selective PARP1 inhibitor is AZD5305.

50. The kit according to either statement 48 or 49, wherein the selective estrogen degrader is camizestrant.

1. A selective PARP1 inhibitor, or a pharmaceutically acceptable salt thereof, for use in the treatment of cancer in a subject, wherein said treatment comprises the separate, sequential or simultaneous administration of i) said selective PARP1 inhibitor, or a pharmaceutically acceptable salt thereof, and ii) a selective estrogen degrader, or a pharmaceutically acceptable salt thereof, to said subject.

2. The selective PARP1 inhibitor, or a pharmaceutically acceptable salt thereof, for use according to claim 1, wherein the cancer is:
a) breast cancer;
b) ER+ breast cancer;
c) ER+ breast cancer which is BRCA1/2m; or
d) ER+ breast cancer which is HR deficient.

3. The selective PARP1 inhibitor, or a pharmaceutically acceptable salt thereof, for use according to either claim 1 or claim 2, wherein the selective PARP inhibitor is AZD5305.

4. The selective PARP1 inhibitor, or a pharmaceutically acceptable salt thereof, for use according to any one of claims 1 to 3, wherein the selective estrogen degrader is camizestrant.

5. The selective PARP1 inhibitor, or a pharmaceutically acceptable salt thereof, for use according to either claim 3 or claim 4, wherein AZD5305 is administered once daily.

6. The selective PARP1 inhibitor, or a pharmaceutically acceptable salt thereof, for use according to claim 5, wherein AZD5305 is administered in a dose of up to about 60 mg per day.

7. The selective PARP1 inhibitor, or a pharmaceutically acceptable salt thereof, for use according to claim 6, wherein AZD5305 is administered in a dose of 60 mg per day.

8. The selective PARP1 inhibitor, or a pharmaceutically acceptable salt thereof, for use according to claim 6, wherein AZD5305 is administered in a dose of 20 mg per day.

9. The selective PARP1 inhibitor, or a pharmaceutically acceptable salt thereof, for use according to any one of claims 4 to 8, wherein the selective estrogen degrader is camizestrant and is administered once daily.

10. The selective PARP1 inhibitor, or a pharmaceutically acceptable salt thereof, for use according to claim 9, wherein camizestrant is administered in a dose of about 50 mg to about 150 mg once a day.

11. The selective PARP1 inhibitor, or a pharmaceutically acceptable salt thereof, for use according to claim 10, wherein camizestrant is administered in a dose of 75 mg once a day.

12. A selective estrogen degrader, or a pharmaceutically acceptable salt thereof, for use in the treatment of cancer in a subject, wherein said treatment comprises the separate, sequential or simultaneous administration of i) said selective estrogen degrader, or a pharmaceutically acceptable salt thereof, and ii) a selective PARP1 inhibitor, or a pharmaceutically acceptable salt thereof, to said subject.

13. The selective estrogen degrader, or a pharmaceutically acceptable salt thereof, for use according to claim 12, wherein the cancer is:
a) breast cancer;
b) ER+ breast cancer;
c) ER+ breast cancer which is BRCA1/2m; or
d) ER+ breast cancer which is HR deficient.

14. The selective estrogen degrader, or a pharmaceutically acceptable salt thereof, for use according to either claim 12 or claim 13, wherein the selective PARP inhibitor is AZD5305.

15. The selective estrogen degrader, or a pharmaceutically acceptable salt thereof, for use according to any one of claims 12 to 14, wherein the selective estrogen degrader is camizestrant.

16. The selective estrogen degrader, or a pharmaceutically acceptable salt thereof, for use according to either claim 14 or claim 15, wherein AZD5305 is administered once daily.

17. The selective estrogen degrader, or a pharmaceutically acceptable salt thereof, for use according to claim 16, wherein AZD5305 is administered in a dose of up to about 60 mg per day.

18. The selective estrogen degrader, or a pharmaceutically acceptable salt thereof, for use according to claim 17, wherein AZD5305 is administered in a dose of 60 mg per day.

19. The selective estrogen degrader, or a pharmaceutically acceptable salt thereof, for use according to claim 17, wherein AZD5305 is administered in a dose of 20 mg per day.

20. The selective estrogen degrader, or a pharmaceutically acceptable salt thereof, for use according to any one of claims 15 to 19, wherein the selective estrogen degrader is camizestrant and is administered once daily.

21. The selective estrogen degrader, or a pharmaceutically acceptable salt thereof, for use according to claim 16, wherein camizestrant is administered in a dose of about 50 mg to about 150 mg once a day.

22. The selective estrogen degrader, or a pharmaceutically acceptable salt thereof, for use according to claim 17, wherein camizestrant is administered in a dose of 75 mg once a day.

23. A pharmaceutical product comprising i) a selective PARP1 inhibitor or a pharmaceutically acceptable salt thereof, and ii) a selective estrogen degrader or a pharmaceutically acceptable salt thereof.

24. The pharmaceutical product according to claim 23, wherein the selective PARP1 inhibitor is AZD5305.

25. The pharmaceutical product according to either claim 23 or 24, wherein the selective estrogen degrader is camizestrant.
